(19) United States
(12) Patent Application Publication
(10) Pub. No.: US 2009/0030381 A1
(43) Pub. Date: Jan. 29, 2009

(54) Arced Hypodermic Needle

(76) Inventors: Casey J. Lind, Irvine, CA (US); Robert J. Sanchez, Oceanside, CA (US); Dyson W. Hickingbotham, Stouchsberg, PA (US)

Correspondence Address: ALCON, IP LEGAL, TB4-8, 6201 SOUTH FREEWAY, FORT WORTH, TX, 76134, US

(21) Appl. No.: 11/781,646

(22) Filed: Jul. 23, 2007

(51) Int. Cl. A61M 5/32

(52) U.S. Cl. 604/274

(57) ABSTRACT

A hypodermic needle for delivering a bolus of a pharmaceutically active agent to the posterior chamber of the eye having a lancet disposed at the distal tip and a lumen disposed along a longitudinal axis within. The lancet may be formed by an angled linear cut coming to a point at the distal tip of the needle, or an arc-shaped cut beginning at the needle tip parallel to the longitudinal axis and gradually arcing upward to roughly perpendicular to the longitudinal axis. Additionally, cuts, grooves, channels, or teeth may be added to the surface of the lancet in a regular or irregular pattern for the purpose of increasing surface tension on the drug bolus.

The present invention relates to a single-use medical device and more particularly to an ophthalmic drug delivery device with an arced hypodermic needle.

### BACKGROUND OF THE INVENTION

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically manually made using a conventional syringe and needle, or an infusion cannula. In the past, if a procedure required that a cannula be inserted into the eye a trocar stick was required to pre-puncture the eye. In many cases, this was accomplished by having the cannula preinstalled around the trocar, so that, once the trocar had punctured the eye, the cannula would be slid into the puncture, and the trocar would then be removed. Problems would arise with respect to conditions requiring a cannula that cannot utilize a trocar due to cannula length or the injection location. In this scenario, the only available alternative is to pre-puncture the eye, remove the trocar, and then attempt to insert the cannula into the puncture wound. This procedure introduces significant risks, including but not limited to trauma to the eye at the incision site, increased risk of post-operative infection, and loss of intra ocular tissue and fluid since the eye is a pressurized globe. In addition, this requirement adds significant time to the overall procedure.

In addition, advancements have been made in the types of drugs used to treat the above mentioned, and other eye diseases. Specifically, several developments have been made utilizing phase transition or reagent/drug mixtures. In this procedure, a drug, or mixture, is heated in a delivery device, such as a syringe and needle, and delivered to the posterior chamber of the eye in a semi solid or liquid state. Once in the posterior chamber of the eye, the drug cools and solidifies. As the solid dissolves, the drug is delivered to the eye over an extended length of time greater than standard liquid injections, thereby reducing the frequency of required treatments. In such a procedure, it is desirable to have the drug introduced into the eye in a roughly spherical shape as a means of maximizing the drug delivery time. Use of prior art needles for such a treatment would result in a cylindrical or “worm shaped” solid being deposited in the eye. Such a shape has considerably greater surface area, and, as such, would dissolve faster eliminating the benefits of time-released delivery.

There are several commercial needle styles involving holes distal from the tip such as the SPROTTE® spinal needle sold by Dyna Medical Corporation of London, Ontario, Canada. Many of these are designed for irrigation and aspiration applications. Of those commercialized, there are none specifically associated with injectors that are designed to deliver phase transition materials in an injectable state (liquid, gel or semi-solid).

Accordingly, it would be desirable to have a hypodermic needle that is able to penetrate the eye without the need of pre-puncturing, allows for a suture-less procedure and produces a roughly spherical bolus upon drug delivery. An arc cut into the distal end of the needle could be used to aid the formation of a spherical bolus. In addition, perpendicular channels cut across the face of the arc could also be used to increase the area of contact between the needle and the bolus thereby increasing the surface tension on the bolus. Such a device provides numerous benefits over prior art needles.

### SUMMARY OF THE INVENTION

In one embodiment, consistent with the principles of the present invention, the present invention is a hypodermic needle, for delivering a bolus of a pharmaceutically active agent to the posterior chamber of the eye, having a lancet disposed at the distal tip and a lumen disposed along a longitudinal axis within. The lancet may be formed by an angled linear cut coming to a point at the distal tip of the needle, or an arc-shaped cut beginning a the needle tip parallel to the longitudinal axis and gradually arcing upward to roughly perpendicular to the longitudinal axis. Additionally, cuts, grooves, channels, or teeth may be added to the surface of the lancet in a regular or irregular pattern for the purpose of increasing surface tension on the drug bolus.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a side cross sectional view of an arced hypodermic needle according to a preferred embodiment of the present invention.

FIG. 2 is a perspective view of an arced hypodermic needle according to a preferred embodiment of the present invention.

FIG. 3 is a cross section view of an arced hypodermic needle according to a preferred embodiment of the present invention engaged in the delivery of a pharmaceutically active agent into the eye.

### DETAILED DESCRIPTION OF THE PREFERRED EMBODIMENTS

Reference is now made in detail to the exemplary embodiments of the invention, examples of which are illustrated in the accompanying figures. Wherever possible, the same reference numbers are used throughout the figures to refer to the same or like parts.

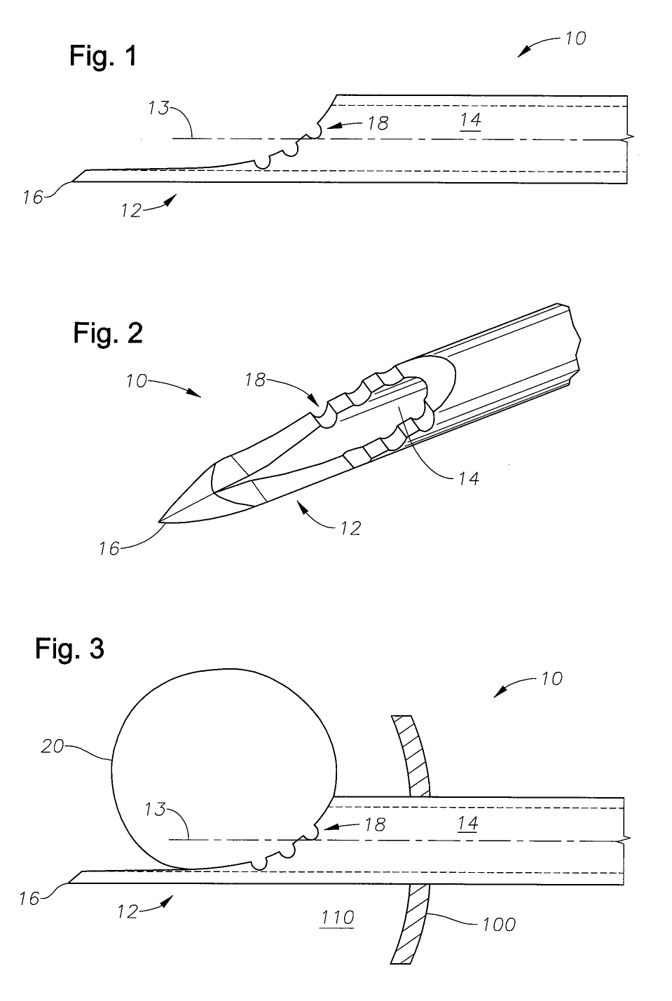

FIGS. 1 and 2 illustrate a hypodermic needle including a lancet, a cannula having an internal lumen, and a plurality of channels according to an embodiment of the present invention. In FIG. 1, needle 10 includes lancet 12 and cannula 11. Lumen 14 is disposed within cannula 11 along longitudinal axis 13. Lancet 12 is located at distal end 16 of cannula 11. Channels 18 are disposed across the surface of lancet 12.

Needle 10 is fluidly connected to any appropriate mechanism for storage and delivery of a pharmaceutically active agent, such as a syringe, cannula, or IV tube (not shown). The pharmaceutically active agent is contained in the storage mechanism, and is typically a drug suspended in a phase transition compound. The phase transition compound is in a solid or semi-solid state at lower temperatures and in a liquid or less viscous state at higher temperatures. Such a compound can be heated to a liquid or less viscous state and injected into the eye, where it forms a bolus that erodes over time.

Needle 10 is adapted to deliver a substance, such as a drug, into an eye. Needle 10 may be of any commonly known configuration, and may be made from any appropriate material, such as surgical stainless steel. Preferably, needle 10 is designed such that its thermal characteristics are conducive to the particular drug delivery application. For example, when a heated drug is to be delivered, needle 10 may be relatively short (several millimeters) in length to facilitate proper delivery of the drug due to thermal characteristics.

Lancet 12 is located at the distal end 16 of cannula 11, and may be formed by any appropriate means such as an angled or arced cut. Most preferably, lancet 12 is formed by an arced cut that begins at the distal end 16 and extends along the length of needle 10 roughly parallel to longitudinal axis 13, and gradually arcing to be roughly perpendicular to longitudinal axis 13. Additionally, channels 18 are disposed on the surface of lancet 12. Channels 18 may have any appropriate cross sectional shape, such as semi-circular, triangular, or rectangular, but are most preferably semi-circular. In addition, channels may be disposed at either regular or irregular intervals along the surface of lancet 12. Finally, opposing sides of channels 18 may be symmetrical or asymmetrical, but are preferably symmetrical.

Lumen 14 is disposed within cannula 11 along longitudinal axis 13. Lumen 14 extends the entire length of needle 10, and is of sufficient diameter to facilitate the delivery of a substance into the eye. For example, lumen 14 may be of any gauge diameter deemed sufficient such as but not limited to 25 gauge or 27 gauge. Lumen 14 terminates in orifice 15 located on the surface of lancet 12. The arc shaped profile of lancet 12 results in orifice 15 having a roughly teardrop or oblong shape.

During operation, lancet 12 is used to pierce sclera 100, as shown in FIG. 3. Needle 10 is then inserted into posterior chamber 110. A phase transition drug formulation heated to a liquid or less viscous state is then gradually expressed from orifice 15 into the eye and cools to form bolus 20. The arc shape of lancet 12 increases the contact area between the drug formulation 20 and lancet 12, helping it to solidify into bolus 20 to take on a roughly spherical shape. Channels 18 serve to further increase the surface tension on bolus 20.

From the above, it may be appreciated that the present invention provides an improved hypodermic needle that is capable of delivering a bolus of a pharmaceutically active agent to the posterior chamber of the eye, and allows for a suture-less procedure. The present invention provides a hypodermic needle having a lancet capable of piercing the sclera of an eye. Channels cut across the lancet increase the surface tension on the pharmaceutically active agent, and aid in the creation of a roughly spherical bolus. The present invention is illustrated herein by example and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A hypodermic needle comprising:
a cannula disposed along a longitudinal axis;
a lancet disposed at a distal end of said cannula, the lancet generally parallel to said longitudinal axis, the lancet having a generally curved profile;
a lumen disposed within said cannula generally parallel to said longitudinal axis; and
a plurality of channels disposed on a surface of said lancet, the plurality of channels disposed generally perpendicular to said longitudinal axis.

2. The needle of claim 1 wherein said cannula further comprises an oblong shaped orifice disposed on a surface of said lancet.

2. The needle of claim 1 wherein at least one of said channels has a semi-circular cross section.

3. The needle of claim 1 wherein at least one of said channels has a triangular cross section.

4. The needle of claim 1 wherein at least one of said channels has a rectangular cross section.

5. The needle of claim 1 wherein said channels are disposed at regular intervals.

6. The needle of claim 1 wherein said channels are disposed at irregular intervals.

7. The needle of claim 1 wherein said channels provide surface tension on a substance to cause the substance to form a sphere or sphere like shape.

8. The needle of claim 1 wherein said curved profile of said lancet provides surface tension on a substance to cause the substance to form a sphere or sphere like shape.

9. The needle of claim 1 wherein said cannula is in the range of 25 to 30 gauge.

10. A hypodermic needle comprising:
a cannula disposed along a longitudinal axis;
a lancet disposed at a distal end of said cannula, the lancet generally parallel to said longitudinal axis, the lancet having a generally curved profile such that an opening at the distal end of said cannula is generally teardrop shaped;
a lumen disposed within said cannula generally parallel to said longitudinal axis; and
a plurality of channels disposed on a surface of said lancet, said plurality of channels disposed generally perpendicular to said longitudinal axis, at least one of said channels having a semi-circular cross section.

11. The needle of claim 10 wherein said channels are disposed at regular intervals.

12. The needle of claim 10 wherein said channels are disposed at irregular intervals.

13. The needle of claim 10 wherein said cannula is in the range of 25 to 30 gauge.

14. A hypodermic needle comprising:
a cannula disposed along a longitudinal axis;
a lancet disposed at a distal end of said cannula, the lancet generally parallel to said longitudinal axis, the lancet having a generally curved profile such that an opening at the distal end of said cannula is generally teardrop shaped;
a lumen disposed within said cannula generally parallel to said longitudinal axis; and
a plurality of channels disposed on a surface of said lancet, said plurality of channels disposed generally perpendicular to said longitudinal axis, at least one of said channels having a triangular cross section.

15. The needle of claim 14 wherein said channels are disposed at regular intervals.

16. The needle of claim 14 wherein said channels are disposed at irregular intervals.

17. The needle of claim 14 wherein said cannula is in the range of 25 to 30 gauge.

18. A hypodermic needle comprising:
a cannula disposed along a longitudinal axis;
a lancet disposed at a distal end of said cannula, the lancet generally parallel to said longitudinal axis, the lancet having a generally curved profile such that an opening at the distal end of said cannula is generally teardrop shaped;
a lumen disposed within said cannula generally parallel to said longitudinal axis; and
a plurality of channels disposed on a surface of said lancet, said plurality of channels disposed generally perpendicular to said longitudinal axis, at least one of said channels having a rectangular cross section.

19. The needle of claim 18 wherein said channels are disposed at regular intervals.

20. The needle of claim 18 wherein said channels are disposed at irregular intervals.
